(19) United States
(12) Patent Application Publication
(10) Pub. No.: US 2016/0274075 A1
(43) Pub. Date: Sep. 22, 2016

(54) SYSTEM AND METHOD FOR CALIBRATING A PARTICULATE MATTER SENSOR

(71) Applicant: Caterpillar Inc., Peoria, IL (US)

(72) Inventor: Xinyu Ge, Peoria, IL (US)

(73) Assignee: Caterpillar Inc., Peoria, IL (US)

(21) Appl. No.: 14/658,317

(22) Filed: Mar. 16, 2015

(51) Int. Cl. G01N 33/00

(52) U.S. Cl. CPC G01N 33/0006; G01N 33/0037

(57) ABSTRACT

A method of calibrating a particulate matter sensor of an engine is provided. The method includes supplying an exhaust from the engine at a predefined particulate matter level to the particulate matter sensor. The method also includes receiving, via the particulate matter sensor, a reading indicative of a particulate matter level in the exhaust. The method further includes calibrating the particulate matter sensor based on the reading and the predefined particulate matter level.

### TECHNICAL FIELD

The present disclosure relates to a method for calibrating a particulate matter sensor, and more specifically to a system and the method for calibrating the particulate matter sensor onboard.

### BACKGROUND

Typically, an engine such as a diesel engine or a dual fuel engine may be associated with an aftertreatment system. The aftertreatment system may be configured to treat and reduce a level of particulate matter or other emissions in an exhaust flow, prior to the exhaust exiting into the atmosphere. In order to reduce the level of particulate matter, the aftertreatment system may include a particulate matter filter such as a Diesel particulate filter (DPF). One or more particulate matter sensors may be located either downstream or upstream to the filter to determine the level of particulate matter in the exhaust. The sensors may also be used to monitor a performance of the filter.

However, various characteristics of the exhaust flow and/or other environmental factors during operation of the engine may affect a functioning of the particulate matter sensors. In an example, the particulate matter sensor may experience many disturbances during measurement. Other measurement noises may also affect the particulate matter sensor's reading such as an extra-large particulate matter passing through or the change of exhaust flow patterns during engine transient events. The noise on the particulate matter sensor signal and/or shifting of particulate matter sensor may interfere with the ability to detect when the filter has failed. In another example, dirt, dust, and/or other debris may also accumulate on or near electrodes of the particulate matter sensor. The accumulation of debris may change the electrical resistance at the electrodes and cause the particulate matter sensor to read a particulate matter level that is not commensurate with the actual particulate matter level in the exhaust.

Under such circumstances, the particulate matter sensors may malfunction or otherwise inaccurately measure and/or indicate particulate matter levels. Such a situation may also effect other operations of the engine and/or the aftertreatment system such as, a regeneration and a diagnosis of the filter and other components.

For reference, U.S. Pat. No. 8,131,495 (hereinafter “the '495 patent”) describes a system that includes a filter, a sensor, a processor, and a memory. The filter can be coupled to an engine exhaust and can operate in an accumulating mode during which particulate matter (PM) from the engine is trapped and also operate in a regenerating mode during which PM in the filter is burnt out. The sensor is coupled to a discharge port of the filter and has an output to provide a sensor signal based on a concentration of PM in the filtered exhaust. The processor is coupled to receive the sensor signal and operable to determine at least one of a base level for the sensor signal during the accumulating mode and a regenerate level for the sensor signal during the regenerating mode, and operable to determine a calibration value for the sensor using at least one of the base level and the regenerate level. The memory stores the calibration value.

The system of the '495 patent is operable to calibrate the base level particulate matter emission as zero level for the particulate matter sensor. However, such a single reading check i.e., a use of the base level particulate matter emission may not provide a comprehensive and accurate calibration for the particulate matter sensor.

### SUMMARY OF THE DISCLOSURE

In one aspect of the present disclosure, a method of calibrating a particulate matter sensor of an engine is provided. The method includes supplying an exhaust from the engine at a predefined particulate matter level to the particulate matter sensor. The method also includes receiving, via the particulate matter sensor, a reading indicative of a particulate matter level in the exhaust. The method further includes calibrating the particulate matter sensor based on the reading and the predefined particulate matter level.

In another aspect of the present disclosure, a controller disposed in communication with a particulate matter (PM) sensor associated with an engine is provided. The controller is configured to determine a predefined particulate matter level of an exhaust from the engine based on a predetermined calibration condition of the engine. The controller is also configured to receive, via the particulate matter sensor, a reading indicative of a particulate matter level in the exhaust and calibrate the particulate matter sensor based on the reading and the predefined particulate matter level.

In yet another aspect of the present disclosure, a system for calibrating a particulate matter sensor associated with an engine is provided. The engine includes an intake manifold, an exhaust manifold and, an Exhaust Gas Recirculation (EGR) conduit fluidly connecting the exhaust manifold and the intake manifold. The system includes a first conduit fluidly connected with the EGR conduit and a first valve unit disposed in the first conduit and configured to selectively allow an exhaust from the EGR conduit to flow therethrough. The system also includes a second valve unit in fluid communication with the first conduit and configured to selectively direct the exhaust from the first conduit to the particulate matter sensor. The system further includes a controller communicably coupled to the first valve unit, the second valve unit and the particulate matter sensor. The controller is configured to regulate the first valve unit and the second valve unit to direct an exhaust at a predefined particulate matter level from the EGR conduit to the particulate matter sensor. The controller is also configured to receive, via the particulate matter sensor, a reading indicative of a particulate matter level in the exhaust. The controller is further configured to calibrate the particulate matter sensor based on the reading and the predefined particulate matter level.

Other features and aspects of this disclosure will be apparent from the following description and the accompanying drawings.

### BRIEF DESCRIPTION OF THE DRAWINGS

FIG. 1 is a schematic view of a system associated with an exemplary engine having a particulate matter sensor, according to an embodiment of the present disclosure;

FIG. 2 is a block diagram for the system, according to an embodiment of the present disclosure;

FIG. 3 is a flow chart for a controller of the system for calibrating the particulate matter sensor, according to an embodiment of the present disclosure; and

FIG. 4 is a flow chart for a method of calibrating the particulate matter sensor of the engine, according to an embodiment of the present disclosure.

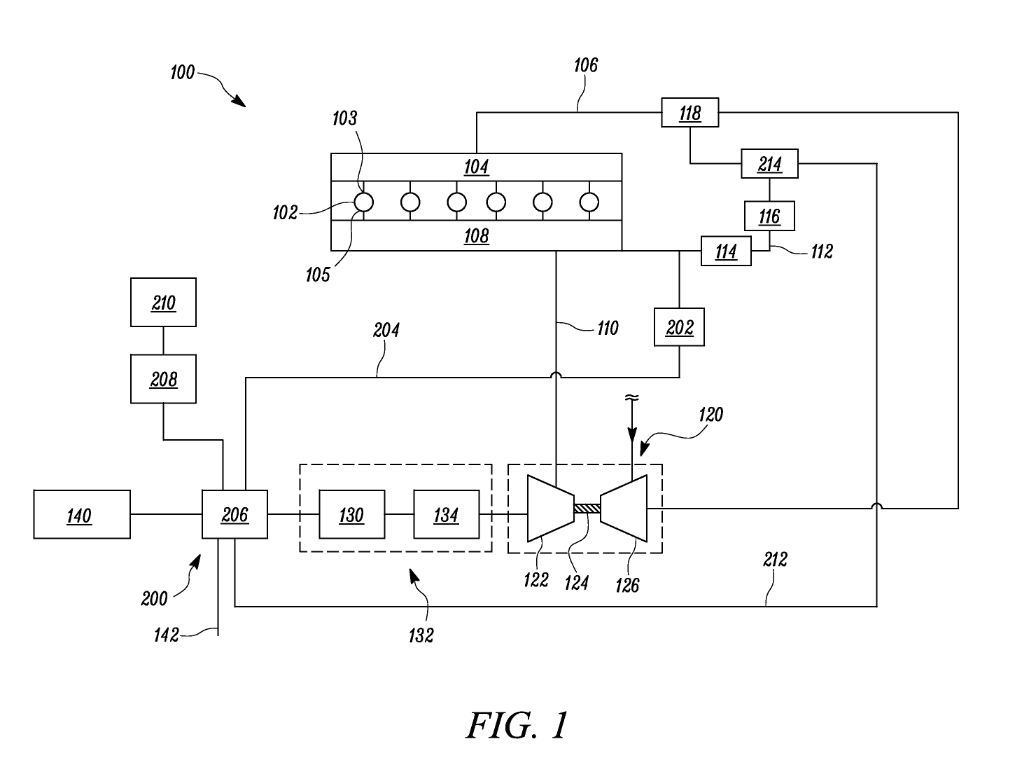

### DETAILED DESCRIPTION

Wherever possible, the same reference numbers will be used throughout the drawings to refer to the same or the like parts. Referring to FIG. 1, a schematic diagram of a system 200 associated with an exemplary engine 100 is illustrated. The engine 100 may embody a combustion engine, such as, a reciprocating piston engine or a gas turbine engine. The engine 100 may be a spark ignition engine or a compression ignition engine, such as, a diesel engine, a homogeneous charge compression ignition engine, or a reactivity controlled compression ignition engine, or other types of engines. The engine 100 may be fueled by gasoline, diesel fuel, biodiesel, dimethyl ether, alcohol, natural gas, propane, hydrogen, combinations thereof, or any other combustion fuel.

The engine 100 may be used to provide power to various types of applications and/or to machines including, but not limited to, an on-highway truck, an off-highway truck, an earth moving machine, an electric generator, a compressor and so on. Accordingly, the engine 100 may be associated with an industry including, but not limited to, transportation, construction, agriculture, forestry, power generation, and material handling.

The engine 100 includes one or more cylinders 102 implemented therein. Although six cylinders 102 are shown, it is recognized that the actual number of cylinders of the engine 100 may vary and that the engine 100 may be of an in-line type, a V-type, a rotary type, and the like. Each of the cylinders 102 may be configured to slidably receive a piston (not shown) therein. The engine 100 may also include one or more fuel injectors or admission valves or a combination thereof for providing fuel to the cylinders 102 that may be used for combustion in the engine 100.

Each of the cylinders 102 may include an intake port 103 and an exhaust port 105 in fluid communication with an intake manifold 104 and an exhaust manifold 108 respectively. The cylinders 102 receive, via the intake port 103, intake air for the combustion from the intake manifold 104. The intake manifold 104 may further be in fluid communication with an intake conduit 106.

The engine 100 also includes an exhaust conduit 110 in fluid communication with the exhaust manifold 108. The exhaust port 105 fluidly connects each of the cylinders 102 to the exhaust manifold 108 of the engine 100 to discharge an exhaust created by the combustion of the fuels from the cylinders 102 via the exhaust conduit 110.

As illustrated in FIG. 1, the engine 100 further includes an exhaust gas recirculation (EGR) conduit 112 for redirecting a portion of the exhaust from the exhaust conduit 110 to the intake manifold 104. The EGR conduit 112 is fluidly connected between the intake manifold 104 and the exhaust manifold 108. Further, an EGR valve 114 may be disposed in the EGR conduit 112 of the engine 100. The EGR valve 114 may be regulated to control an amount of the exhaust to be passed to the intake manifold 104 via the EGR conduit 112. The engine 100 may further include an EGR cooler 116 disposed in the EGR conduit 112. The EGR cooler 116 may be provided to reduce a temperature of the exhaust passing through the EGR conduit 112.

The engine 100 may further include a turbocharger 120 including a turbine 122 and a compressor 126 connected to each other by a shaft 124. The turbine 122 may be configured to be driven by the exhaust received from the exhaust conduit 110 causing the shaft 124 to rotate, thereby driving the compressor 126. The compressor 126, in turn, may draw in intake air via an inlet (not shown) and compress the intake air. The compressed air may be directed to the intake manifold 104. Alternatively, the turbine 122 may be driven by other power sources such as a motor to subsequently drive the compressor 126.

The engine 100 may further include a mixing system 118 fluidly communicating the intake conduit 106 and the EGR conduit 112. The mixing system 118 may be positioned downstream of the EGR cooler 116 and upstream of the intake manifold 104. The mixing system 118 may include a one or more mixing elements such as, flapper mixers, swirl mixers, impingement mixers, and the like. The mixing elements may be configured to combine and/or mix the exhaust from the EGR conduit 112 with the intake air from the intake conduit 106 to form a combustion gas for the cylinders 102.

The engine 100 may include other components (not shown), for example, a fuel system, a lubrication system, a cooling system, actuators or any other appropriate components.

The engine 100 includes an aftertreatment system 132 fluidly connected to the exhaust conduit 110 of the engine 100. The aftertreatment system 132 is configured to treat the exhaust in the exhaust conduit 110 of the engine 100. The exhaust contains emission compounds that may include oxides of nitrogen (NOx), unburned hydrocarbons, particulate matter, and/or other combustion products. The aftertreatment system 132 may be configured to trap or convert NOx, unburned hydrocarbons, particulate matter, combinations thereof, or other combustion products present in the exhaust gas flow, before exiting the engine 100.

In the illustrated embodiment, the aftertreatment system 132 includes a filter module 130 that is fluidly connected to the exhaust conduit 110 of the engine 100. The filter module 130 is configured to remove particulate matter, such as soot, or other Soluble Organic Fraction (SOF) of particulate matter from the exhaust of engine 100. The filter module 130 may include a filter medium, e.g., a metal mesh or screen, a porous ceramic material, such as cordierite, or another medium to remove (i.e., trap) one or more types of particulate matter from the exhaust. Additionally, the filter medium may be coated with a suitable catalyst to promote oxidation of any particulate matter in the exhaust that may be trapped in the filter medium. However, it may be recognized that the filter module 130 may be of any other type configured to reduce the particulate matter.

The aftertreatment system 132 may also include a regeneration mechanism (not shown) configured to regenerate the filter module 130. The regeneration mechanism may embody any suitable technique such as, a dosing system or a backpressure-generating flow restrictor to suitably regenerate the filter module 130. In an example, the filter module 130 may be regenerated when an amount of trapped particulate matter in the filter medium exceeds a threshold value.

The aftertreatment system 132 may further include additional components such as, a Selective Catalytic Reduction (SCR) module 134 provided upstream of the filter module 130. The SCR module 134 is configured to reduce a concentration of NOx in the exhaust. The SCR module 134 may include a catalyst for facilitating the reaction, reduction, or removal of NOx from the exhaust passing through the SCR module 134. The SCR module 134 may have a honeycomb or other structure made from or coated with an appropriate material. The material may be an oxide, such as vanadium oxide or tungsten oxide, coated on an appropriate substrate, such as titanium dioxide. Other components such as, ammonia oxidation catalyst may also be used to convert any ammonia slip in the exhaust flow from the SCR module 134.

Further, the aftertreatment system 132 may also include a reductant module (not shown) configured to dispense a reductant into the exhaust flow. The reductant may be a fluid, such as, a Diesel Exhaust Fluid (DEF), and may include urea, ammonia, or other reducing agents.

Alternatively, the aftertreatment system 132 may omit the SCR module 134 and include only the filter module 130. In an example, the filter module 130 may alternatively or additionally include multifunctional devices, such as, for example, a catalytic converter and particulate trap combination or a catalytic particulate trap such as, a combined PM/SCR catalyst (not shown) may be used.

The aftertreatment system 132 disclosed herein is provided as a non-limiting example. It will be appreciated that the aftertreatment system 132 may be disposed in various arrangements and/or combinations relative to the exhaust manifold 108. These and other variations in the aftertreatment system 132 design are possible without deviating from the scope of the disclosure.

One or more particulate matter sensors 140 may be associated with the engine 100 and configured to determine a particulate matter level in an exhaust supplied thereto. In the illustrated embodiment, the particulate matter sensor 140 is disposed downstream of the filter module 130. Accordingly, the exhaust from the filter module 130 may be passed to the particulate matter sensor 140 that is configured to determine a particulate matter level in the exhaust. In one example, the particulate matter level may be indicative of concentration of soot in the exhaust. Additionally, it may be contemplated to provide another particulate matter sensor 140 upstream to the filter module 130.

In an embodiment, the particulate matter sensor 140 may be a resistivity and/or a conductivity based sensor. For example, the sensor may include two electrodes (not shown). A resistance between the electrodes may change based on an accumulation of the particulate matter. The resistance may be measured with a variety of techniques. In an example, a bias voltage and/or current may be applied to the particulate matter sensor 140, a resistor network including the sensor, or the like. Further, a voltage on a node of the resistor network may be measured to sense the resistance or conductance of the particulate matter sensor 140. Although a resistivity based sensor has been described, other types of sensor architecture and the associated measurement techniques may be used, such as a capacitance based sensor, optical based sensor, or the like.

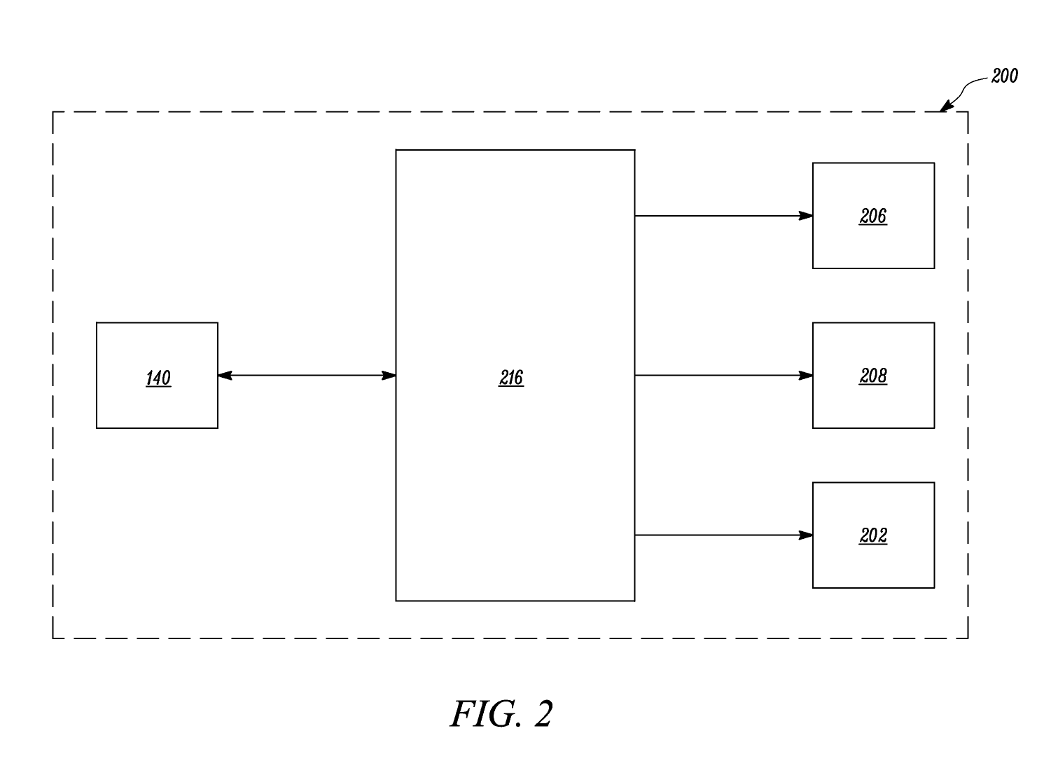

Referring still to FIGS. 1 and 2, a system 200 may be implemented with the engine 100 for calibrating the particulate matter sensor 140. The system 200 includes a first conduit 204 configured to be fluidly connected with the EGR conduit 112. The system 200 also includes a first valve unit 202 disposed in the first conduit 204 and configured to selectively allow the exhaust from the EGR conduit 112 to flow therethrough.

In an embodiment, the first valve unit 202 may be a configured to be operable in an open position and a closed position. In the open position, the first valve unit 202 may allow the exhaust from the EGR conduit 112 to flow therethrough to the first conduit 204. In the closed position, the first valve unit 202 may block the fluid communication between the exhaust conduit 110 and the first conduit 204. In an example, the first valve unit 202 may embody a shut-off valve.

The system 200 also includes a second valve unit 206 in fluid communication with the first conduit 204. The second valve unit 206 is configured to selectively direct the exhaust from the first conduit 204 to the particulate matter sensor 140. The second valve unit 206 may also be fluidly coupled to an exit 142.

In the illustrated embodiment, the system 200 further includes a third valve unit 208 in fluid communication with an air source 210 and the second valve unit 206. The third valve unit 208 is configured to selectively allow the air from the air source 210 to flow therethrough. In one example, the air source 210 may be a centralized tank. As such, the air source 210 may provide the air at a regulated pressure to the third valve unit 208. Alternatively, the air may be an ambient air. In an embodiment, the third valve unit 208 may be configured to be operable in an open position and a closed position. In the open position, the third valve unit 208 may allow the air from the air source 210 to flow therethrough to the second valve unit 206. In the closed position, the third valve unit 208 may block the fluid communication between the air source 210 and the second valve unit 206. In an example, the third valve unit 208 may embody a shut-off valve.

The second valve unit 206 may further be configured to selectively allow the air from the third valve unit 208 to flow to the particulate matter sensor 140. Further, the system 200 may also include a second conduit 212 fluidly connecting with the second valve unit 206. The second conduit 212 may be configured to be in fluid communication with the intake conduit 106. As shown, the second conduit 212 may be connected between the mixing system 118 and the EGR cooler 116 at a T-connection 214. The second valve unit 206 may be configured to selectively direct the air and/or the exhaust after passing to the particulate matter sensor 140 to the second conduit 212.

The second valve unit 206 disclosed herein may be any type of a valve or valve assemblies configured to selectively effect fluid communication between the first conduit 204 and the second conduit 212, or between the third valve unit 208 and the second conduit 212. For example, the second valve unit 206 may operate in one of a first configuration, a second configuration and a third configuration. In the first configuration, the second valve unit 206 effects fluid communication between the first conduit 204 and the second conduit 212 to allow the exhaust to pass through the particulate matter sensor 140. Further, in the first configuration, the second valve unit 206 blocks the fluid communication between the filter module 130 and the exit 142; and the third valve unit 208 and the second conduit 212. In such a case, the exhaust from the filter module 130 may be directly emitted to the atmosphere in the first configuration.

In the second configuration, the second valve unit 206 effects fluid communication between the third valve unit 208 and the second conduit 212 to allow the air to pass through the PM sensor 140. Accordingly, the second valve unit 206 blocks both the fluid communication between the filter module 130 and the exit 142; and between the first conduit 204 and the second conduit 212. In such a case, the exhaust from the filter module 130 may be directly emitted outside the engine 100 to the atmosphere in the second configuration.

In the third configuration, the second valve unit 206 effects the fluid communication between the filter module 130 and the PM sensor 140 to direct a portion of the exhaust from the filter module 130 to the second valve unit 206, and then to the exit 142. Other portion of the exhaust may be directly emitted to the atmosphere after passing through the filter module 130. Further, in the third configuration, the second valve unit 206 blocks both the fluid communication between the third valve unit 208 and the second conduit 212 and; the first conduit 204 and the second conduit 212.

In one example, the second valve unit 206 may include a 5-way, 3-position valve. In another example, the second valve unit 206 may include a set of valves configured to function as described above.

Referring to FIG. 2, a controller 216 may be implemented in the system 200 for calibrating the particulate matter sensor 140. The controller 216 may include any means for receiving engine operating parameter-related information and/or for monitoring, recording, storing, indexing, processing, and/or communicating such information. These means may include components such as, for example, a memory (not shown), one or more data storage devices, a central processing unit (CPU), or any other components that may be used to run an application.

Although aspects of the present disclosure may be described generally as being stored in the memory (not shown), one skilled in the art will appreciate that these aspects can be stored on or read from types of computer program products or computer-readable media, such as computer chips and secondary storage devices, including hard disks, floppy disks, optical media, CD-ROM, or other forms of RAM or ROM. Various other circuits may be associated with the controller 216, such as power supply circuitry, signal-conditioning circuitry, solenoid driver circuitry, communication circuitry, and other appropriate circuitry.

It should be appreciated that the controller 216 may alternatively include multiple controllers (not shown), each dedicated to perform one or more of these or other functions. Such multiple controllers may be configured to communicate and cooperate with one another. Further, the controllers may also communicate with an engine controller module (ECM) that performs processing and controlling functions such as engine management and control, among other things. In an example, the controller 216 may provide instructions to the ECM to perform one or more of the functions described above. In another example, the controller 216 may also receive, via the ECM, an engine operating parameter-related information as processed by the ECM.

In the illustrated embodiment of FIG. 2, the controller 216 may be communicably coupled to the first, second and third valve units 202, 206, 208. Further, the controller 216 may be configured to operate each of the first, second and third valve units 202, 206, 208 in various configurations as needed. The controller 216 may actuate each of the first, second and third valve units 202, 206, 208 via an electrical actuator, such as a solenoid, a pneumatic actuator, a hydraulic actuator, and the like. In an alternative embodiment, actuation of the first, second and third valve units 202, 206, 208 between various configurations as discussed above may be done manually.

The controller 216 is also disposed in communication with the particulate matter sensor 140 to receive readings indicative of a particulate matter level. The controller 216 may also be configured to operate the particulate matter sensor 140 in a calibration mode and a monitor mode. In the monitor mode, the controller 216 may be configured to suitably operate the first, second and third valve units 202, 206 and 208 to obtain the third configuration as described above. As such, in the monitor mode, a portion of the exhaust may be directed from the filter module 130 to the second valve unit 206, and then to the exit 142 while the other portion of the exhaust may be directly emitted to the atmosphere after passing through the filter module 130.

The controller 216 may be configured to close EGR valve 114 and open the first and third valve units 202, 206 to operate the particulate matter sensor 140 in the calibration mode. Additionally, the controller 216 may also be configured to operate the engine 100 at multiple predetermined calibration conditions in the calibration mode to obtain the exhaust at discrete predefined particulate matter levels at each of these predetermined calibration conditions. The predetermined calibration conditions may be obtained by controlling a specific set of calibration parameters. The predefined set of calibration parameters may include an injection timing, an injection pressure, an injection mode and a fuel quantity.

The controller 216 may refer to engine calibrations maps or reference tables to obtain predetermined calibration points corresponding to the set of calibration parameters. These calibration maps or reference tables may be set during the engine calibration process and stored in the ECM of the engine 100. The controller 216 may be configured to determine that the engine 100 is at the predetermined calibration condition when the measured calibration parameters are substantially close to the corresponding predetermined calibration points.

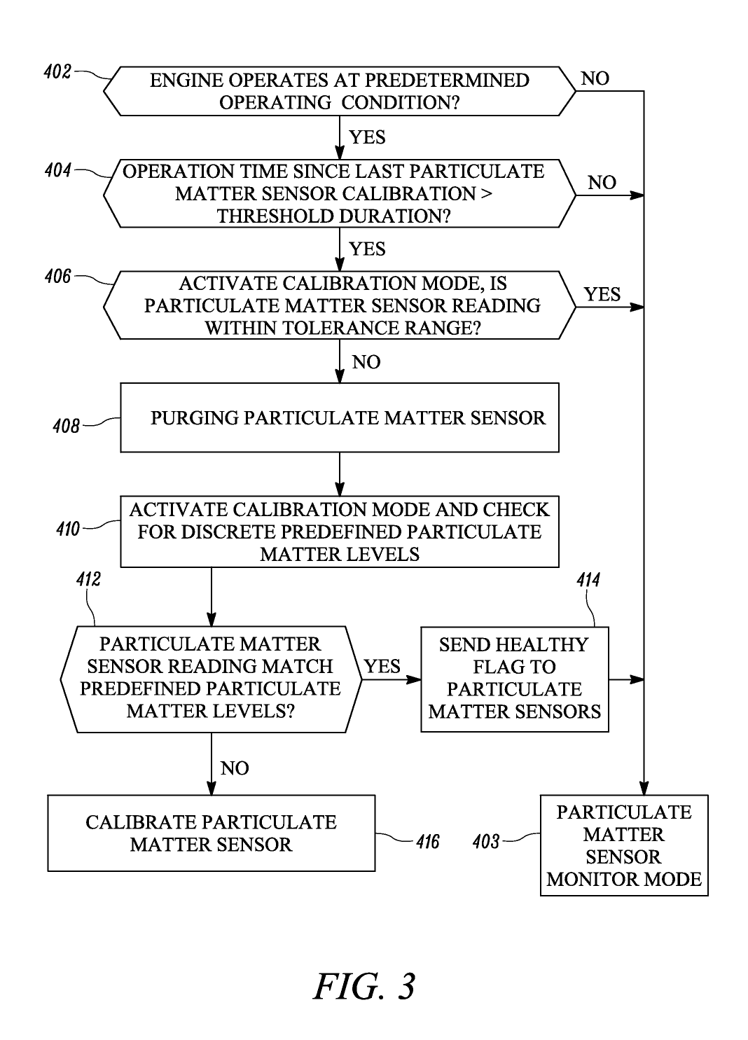

Referring to FIG. 3, an exemplary flowchart implemented by the controller 216 for calibrating the particulate matter sensor 140 is illustrated. At step 402, the controller 216 may determine if the engine 100 is operating in a predetermined operating condition. In an embodiment, the predetermined operating condition may correspond to any one of predefined opportunities at which the calibration mode for the particulate matter sensor 140 may be activated. In an example, the predefined opportunity may occur when the EGR valve 114 is normally closed or partially closed and, when the first and third valve units 202, 206 are in the open position. Further, the controller 216 may also check if a power of the engine 100 may not significantly degrade with the set of calibration parameters utilized to provide the predefined particulate matter levels in the exhaust when the calibration mode for the sensor 140 is activated to determine the predefined opportunity.

In another example, the controller 216 may determine a full load or a part load operating condition as the predetermined operating condition for the engine 100. In yet another example, the controller 216 may determine an idling state of the engine 100 as the predetermined operating condition. The controller 216 is configured to determine if the engine 100 operates at the predetermined operating condition that corresponds to the predefined opportunity to activate the calibration mode.

The controller 216 may pass the control to step 403 if it is determined that the engine 100 is not operating at the predetermined operating condition. At step 403, the controller 216 may operate the particulate matter sensor 140 in the monitor mode. At step 404, the controller 216 may determine if an operating time for the engine 100 since last calibration for the particulate matter sensor 140 exceeds a threshold duration. The controller 216 may pass the control from step 404 to step 406, if the operating time exceeds the threshold duration. However, if the operating time is less than the threshold duration, the controller 216 may pass the control to step 403.

At step 406, the controller 216 is configured to activate the calibration mode for the particulate matter sensor 140. Moreover, the controller 216 may switch an operation of the engine 100 to the predetermined calibration conditions when the engine 100 is operating at the predetermined operating condition for a smooth transition. As described above, the controller 216 may activate the calibration mode by closing EGR valve 114 and opening the first and third valve units 202, 206. Further, in the calibration mode, the engine 100 may generate the exhaust at discrete predefined particulate matter levels corresponding to each of the predetermined calibration conditions. In one example, the controller 216 may determine the particulate matter level based on parameters such as, the injection pressure, the injection timing and the injection mode for the fuel used for combustion in the engine 100. The predefined particulate matter levels corresponding to these predetermined calibration conditions may be stored in the memory associated with the controller 216.

The controller 216 is further configured to selectively operate the first, second and third valve units 202, 206, 208 to supply the exhaust from the engine 100 operating at each of the predetermined calibration conditions to the particulate matter sensor 140. The controller 216 may be configured to control the first second and third valve units 202, 206, 208 to operate in the first configuration. As described above, in the first configuration, the exhaust may be allowed to pass through the particulate matter sensor 140 by effecting the fluid communication between the first conduit 204 and the second conduit 212; and blocking the fluid communication between the filter module 130 and the exit 142, and the third valve unit 208 and the second conduit 212.

The controller 216 is further configured to receive, via the particulate matter sensor 140, a reading indicative of a particulate matter level in the exhaust. As shown, at step 406, the controller 216 may determine if a difference between the reading and the predefined particulate matter level is within a tolerance range. The tolerance range may depend on a variety of characteristics, e.g., the type of machine, the type of engine, the type of fuel used, the layout and arrangement of the after treatment system 132, the filter medium, the sensitivity and/or accuracy of the particulate matter sensor 140 and/or other factors. As such, the tolerance range may be selected based on experimental or simulation data gathered for the particular machine, engine, and a particulate matter sensor 140 strategy employed.

The controller 216 may pass the control from step 406 to step 403, if the difference falls within the tolerance range. Moreover, if at step 404, it is determined that the difference falls out of the tolerance range, the controller 216 may pass the control to step 408.

At step 408, the controller 216 is configured to perform the purging operation on the particulate matter sensor 140. Accordingly, the controller 216 may be configured to selectively operate the first, second and the third valve units 202, 206, 208 to purge the particulate matter sensor 140 with a supply of air during the purging operation. Specifically, the controller 216 may actuate the third valve unit 208 to the open position during a purging operation. The third valve unit 208 in the open position may open the fluid communication between the second valve unit 206 and the air source 210. The controller 216 may actuate the second valve unit 206 to operate in the second configuration to allow the air to flow from the third valve unit 208 to the particulate matter sensor 140. Further, the controller 216 may also actuate the first valve unit 202 to the closed position.

Additionally, during the purging operation, the controller 216 may also be configured to receive, via the particulate matter sensor 140, a reference reading indicative of the particulate matter level in the air. Further, the controller 216 is configured to calibrate the particulate matter sensor 140 by adjusting the reference reading to indicate a zero particulate matter level. However, it may also be envisioned to perform the purging operation at other steps of the calibration mode.

At step 410, the controller 216 may activate the calibration mode for the particulate matter sensor 140. Further at step 410, the controller may operate the engine 100 at one or more of the predetermined calibration conditions in the calibration mode. In an embodiment, the predetermined calibration condition for the engine 100 may be achieved by controlling the calibration parameters such as, the injection pressure, the injection timing, the injection mode and the fuel quantity used. The controller 216 may be further configured to determine predefined particulate matter levels at each of these predetermined calibration conditions.

In an example, a first predetermined calibration condition may correspond to the exhaust having the predefined particulate matter level of about 0.1+/−0.02 g/kw-hr, a second predetermined calibration condition may correspond to the exhaust having the predefined particulate matter level of about 0.2+/−0.02 g/kw-hr and a third predetermined calibration condition may correspond to the exhaust having the predefined particulate matter level of about 0.3+/−0.02 g/kw-hr. These predefined particulate matter levels may be determined during engine calibration process and stored in the memory. Accordingly, the controller 216 may be configured to operate the engine 100 at one or more of these predetermined calibration conditions.

The controller 216 may further supply the exhaust generated by operating the engine 100 in at least one the predetermined calibration conditions to the particulate matter sensor 140. In an embodiment, the controller 216 may further selectively operate the first, second and third valve units 202, 206, 208 to supply the exhaust at the predetermined calibration condition to the particulate matter sensor 140. The controller 216 is also configured to receive readings indicative of the particulate matter level in the exhaust from the particulate matter sensor 140 at the corresponding predetermined calibration conditions.

At step 412, the controller 216 may determine if the readings matches with the corresponding predefined particulate matter levels for the exhaust. The controller 216 may pass the control to step 414, if the readings match the corresponding particulate matter levels. At step 414, the controller 216 may determine the particulate matter sensor 140 as healthy. Further, the controller 216 may also send a flag to the particulate matter sensor 140 indicating that the particulate matter sensor 140 is healthy.

However, if at step 412, it is determined that at least one of the readings does not match with the corresponding particulate matter levels, the controller 216 may pass the control to step 416. At step 416, the controller 216 may calibrate the particulate matter sensor 140 based on the readings and the predefined particulate matter levels. In an example, a linearization method may be used to calibrate the particulate matter sensor 140 based on the set of readings and the set of predefined particulate matter levels. Further, at step 416, the controller 216 may generate a flag indicating that the particulate matter sensor 140 is calibrated.

Additionally, at step 416, the controller 216 may determine if the particulate matter sensor 140 may not be calibrated based on a difference between the readings and the corresponding particulate matter levels. For example, the controller 216 may determine if any of the differences exceeds a threshold difference. In such a case, the controller 216 may send a flag indicating that the particulate matter sensor 140 is faulty if it is determined that the particulate matter sensor 140 may not be calibrated.

In an embodiment, the controller 216 may also be configured to determine an amount of NOx in the exhaust. In an example, the amount of NOx may be determined from a NOx sensor associated with the SCR module 134. Further, the controller 216 may determine an estimated particulate matter level in the exhaust based on the amount of NOx. The controller 216 may refer to reference maps or loop up tables or a mathematical relation to determine the estimated particulate matter level based on the amount of NOx. The controller 216 may be configured to calibrate the particulate matter sensor 140 further based on the estimated particulate matter level based on the amount of NOx.

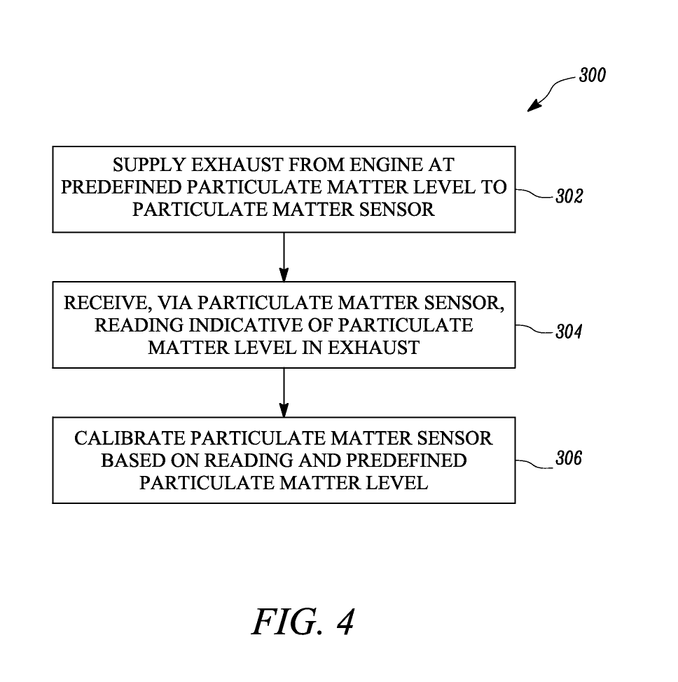

Referring to FIG. 4, a method 300 of calibrating the particulate matter sensor 140 of the engine 100 is illustrated. In an embodiment, one or more steps of the method may be implemented by the control system 200. However, it may also be envisioned to use other alternative configurations of the control system 200 to implement the method 300.

At step 302, the method 300 includes supplying the exhaust at the predefined particulate matter level from the engine 100 to the particulate matter sensor 140. The predefined particulate matter level corresponds to the predetermined calibration condition of the engine 100. Moreover, the predetermined calibration condition corresponds to a calibration condition for the engine 100 at which a particulate matter level in the exhaust may be precisely determined as a function of known calibration parameters of the engine 100.

In an embodiment, the method 300 may also include operating the engine 100 at one of the predetermined calibration conditions. The predetermined calibration condition may be determined based on the calibration parameters such as, the injection timing, the injection pressure and the injection mode for a fuel used for combustion in the engine 100. Moreover, an operation of the engine 100 may be switched to the predetermined calibration conditions when the engine 100 is operating at the predetermined operating condition for smooth transition. As described above, the first, second and third valve units 202, 206, 208 may be suitably controlled to operate in the first configuration so as to supply the exhaust at the predefined particulate matter level to the particulate matter sensor 140.

At step 304, the method 300 includes receiving, via the particulate matter sensor 140, a reading indicative of a particulate matter level in the exhaust. At step 306, the method 300 includes calibrating the particulate matter sensor 140 based on the reading and the predefined particulate matter level. In an embodiment, the controller 216 is configured to repeat the steps 302 and 304 for multiple predetermined calibration conditions to obtain a set of readings corresponding to a set of predefined particulate matter levels. Further, the controller 216 may calibrate the particulate matter sensor 140 based on the set of readings and the set of predefined particulate matter levels using suitable methods e.g., linearization.

The method 300 may also include purging the particulate matter sensor 140 with a supply of air. In an embodiment, as described above, the first, second and third valve units 202, 206, 208 may be suitably controlled to operate in the second configuration so as to supply the air to the particulate matter sensor 140. In one example, the air may be an ambient air. In another example, the air may be supplied from a centralized tank or a purging system.

The method 300 may further include receiving, via the particulate matter sensor 140, a reference reading indicative of a particulate matter level in the supplied air. Accordingly, the method 300 may also include calibrating the particulate matter sensor 140 by adjusting the reference reading to indicate the zero particulate matter level. In other examples, the reference reading may be adjusted to other base readings to suit a specific configuration for the particulate matter sensor 140.

In an embodiment, the method 300 may include determining the estimated particulate matter level in the exhaust at the predetermined calibration condition based on a relationship with the amount of NOx in the exhaust. The method 300 may further include calibrating the particulate matter sensor 140 based on the reading and the estimated particulate matter level in the exhaust.

### INDUSTRIAL APPLICABILITY

With use of the system 200 and the method 300 of the present disclosure, the particulate matter sensor 140 may be calibrated onboard. The controller 216 of the system 200 is configured to periodically calibrate the sensor 140. Moreover, the system 200 includes the valve units 202, 206, 208 that may be removably attached to corresponding components of the engine 100 as needed. Moreover, the system 200 may also include the second conduit 212 through which the exhaust may be re-directed from the particulate matter sensor 140 to the intake manifold 104. As such, the exhaust with high concentrations of particulate matter i.e., high particulate matter level may not be exited to the atmosphere.

The method 300 may further include purging the sensor 140 with a supply of the air. With such an operation, dirt, dust, and/or other debris that may be accumulated inside the sensor 140 may be removed. Further, a zero-reading checking may also be performed during the purging operation.

The system 200 and the method 300 of the present disclosure may also be used to detect when the particulate matter sensor 140 has malfunctioned and is indicating an inaccurate particulate matter level. Further, other appropriate system reactions, such as, triggering an alert, logging a fault code, terminating and/or precluding future regeneration events, precluding exhaust or aftertreatment system diagnostic tests may be performed by the controller 216 in response to a detected malfunction of the particulate matter sensor 140. As such, the effects of operating the engine 100 and the associated machine in the undesirable state may be reduced or avoided. Specifically, improper operation of the aftertreatment system 132, e.g., excessive or insufficient regeneration events, failure of the exhaust treatment device, decreased fuel efficiency, and/or excess emissions may be avoided.

While aspects of the present disclosure have been particularly shown and described with reference to the embodiments above, it will be understood by those skilled in the art that various additional embodiments may be contemplated by the modification of the disclosed machines, systems and methods without departing from the spirit and scope of what is disclosed. Such embodiments should be understood to fall within the scope of the present disclosure as determined based upon the claims and any equivalents thereof.

What is claimed is:

1. A method of calibrating a particulate matter sensor associated with an engine, the method comprising:
supplying an exhaust from the engine at a predefined particulate matter level to the particulate matter sensor;
receiving, via the particulate matter sensor, a reading indicative of a particulate matter level in the exhaust; and
calibrating the particulate matter sensor based on the reading and the predefined particulate matter level.

2. The method of claim 1, wherein the predefined particulate matter level corresponds to a predetermined calibration condition of the engine.

3. The method of claim 2, wherein the predetermined calibration condition is determined based at least on an injection timing, an injection pressure and an injection mode for a fuel used for combustion in the engine.

4. The method of claim 1 further comprising supplying air to the particulate matter sensor during a purging operation.

5. The method of claim 4 further comprising:
receiving, via the particulate matter sensor, a reference reading indicative of a particulate matter level in the air during the purging operation; and
calibrating the particulate matter sensor by adjusting the reference reading to indicate a zero particulate matter level.

6. The method of claim 4 further comprising regulating a first valve unit and a second valve unit to supply the exhaust at the predefined particulate matter level from the engine to the particulate matter sensor, wherein the first valve unit is disposed in fluid communication with an Exhaust Gas Recirculation (EGR) conduit of the engine; and wherein the second valve unit is in fluid communication with the first valve unit and the particulate matter sensor.

7. The method of claim 6 further comprising regulating the second valve unit to selectively supply the air to the particulate matter sensor.

8. The method of claim 2 further comprising:
determining an estimated particulate matter level in the exhaust at the predetermined calibration condition based on a relationship with an amount of NOx in the exhaust; and
calibrating the particulate matter sensor further based on the reading and the estimated particulate matter level in the exhaust.

9. A controller disposed in communication with a particulate matter sensor associated with an engine, the controller configured to:
determine a predefined particulate matter level of an exhaust from the engine corresponding to a predetermined calibration condition of the engine;
receive, via the particulate matter sensor, a reading indicative of a particulate matter level in the exhaust; and
calibrate the particulate matter sensor based on the reading and the predefined particulate matter level.

10. The controller of claim 9, wherein the predetermined calibration condition is determined based on an injection timing, an injection pressure and an injection mode for a fuel used for combustion in the engine.

11. The controller of claim 9 further configured to selectively operate the engine to supply air to the particulate matter sensor during a purging operation.

12. The controller of claim 11 further configured to:
receive, via the particulate matter sensor, a reference reading indicative of a particulate matter level in the air during the purging operation; and
calibrate the particulate matter sensor by adjusting the reference reading to indicate a zero particulate matter level.

13. The controller of claim 11 further configured to regulate a first valve unit and a second valve unit to supply the exhaust at the predefined particulate matter level from the engine to the particulate matter sensor, wherein the first valve unit is disposed in fluid communication with an Exhaust Gas Recirculation (EGR) conduit of the engine, and wherein the second valve unit is in fluid communication with the first valve unit and the particulate matter sensor.

14. The controller of claim 13 further configured to regulate the second valve unit to selectively supply the air to the particulate matter sensor.

15. The controller of claim 9 further configured to:
determine an estimated particulate matter level in the exhaust at the predetermined calibration condition based on a relationship with an amount of NOx in the exhaust; and
calibrate the particulate matter sensor further based on the reading and the estimated particulate matter level in the exhaust.

16. A system for calibrating a particulate matter sensor associated with an engine having an intake manifold, an exhaust manifold and, an Exhaust Gas Recirculation (EGR) conduit fluidly connecting the exhaust manifold and the intake manifold, the system comprising:
a first conduit fluidly connected with the EGR conduit;
a first valve unit disposed in the first conduit and configured to selectively allow an exhaust from the EGR conduit to flow therethrough;
a second valve unit in fluid communication with the first conduit, the second valve unit configured to selectively direct the exhaust from the first conduit to the particulate matter sensor; and
a controller communicably coupled to the first valve unit, the second valve unit and the particulate matter sensor, the controller configured to:
regulate the first valve unit and the second valve unit to direct the exhaust at a predefined particulate matter level from the EGR conduit to the particulate matter sensor;
receive, via the particulate matter sensor, a reading indicative of a particulate matter level in the exhaust; and
calibrate the particulate matter sensor based on the reading and the predefined particulate matter level.

17. The system of claim 16, wherein the controller is configured to direct the exhaust at the predefined particulate matter level during a predetermined calibration condition of the engine, wherein the predetermined calibration condition is determined based on an injection timing, an injection pressure and an injection mode for a fuel used for combustion in the engine.

18. The system of claim 16 further comprising a third valve unit in fluid communication with an air source and the second valve unit, the third valve unit configured to selectively allow the air from the air source to flow therethrough.

19. The system of claim 18, wherein the controller is further configured to selectively operate the second valve unit and the third valve unit to direct the air from the air source to the particulate matter sensor during a purging operation.

20. The system of claim 19, wherein the controller is further configured to:
receive, via the particulate matter sensor, a reference reading indicative of a particulate matter level in the air during the purging operation; and
calibrate the particulate matter sensor by adjusting the reference reading to indicate the zero particulate matter level.
